(19) United States
(12) Patent Application Publication
(10) Pub. No.: US 2021/0355138 A1
(43) Pub. Date: Nov. 18, 2021

(54) AMINOTHIAZINIES AND THEIR USE AS BACE1 INHIBITORS

(71) Applicant: Eli Lilly and Company, Indianapolis, IN (US)

(72) Inventor: Steven James GREEN, Indianapolis, IN (US)

(21) Appl. No.: 16/319,149

(22) PCT Filed: Aug. 2, 2017
(86) PCT No.: PCT/US2017/045079

§ 371 Date: Jan. 18, 2019

Related U.S. Application Data
(60) Provisional application No. 62/373,459, filed on Aug. 11, 2016.

(51) Int. Cl. C07D 513/04

(52) U.S. Cl. CPC C07D 513/04

(57) ABSTRACT

The present invention provides a compound of Formula I: or a pharmaceutically acceptable salt thereof, and the use of compounds of Formula I for treatment of neurodegenerative diseases and disorders, such as Alzheimer's disease.

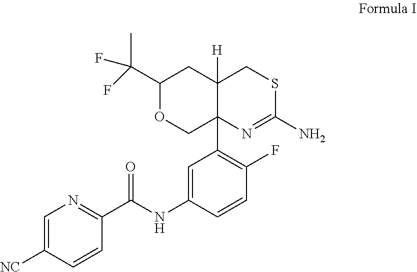 [SMILES: CC(F)(F)C1CC2CSC(N)=NC2(c2cc(NC(=O)c3ccc(C#N)cn3)ccc2F)CO1]

The present invention relates to novel compounds, their use as BACE1 inhibitors, to pharmaceutical compositions comprising the compounds, to methods of using the compounds to treat physiological disorders, and to intermediates and processes useful in the synthesis of the compounds.

The present invention is in the field of treatment of Alzheimer's disease and other diseases and disorders involving amyloid β (Abeta) peptide, a neurotoxic and highly aggregatory peptide segment of the amyloid precursor protein (APP). Alzheimer's disease is a devastating neurodegenerative disorder that affects millions of patients worldwide. In view of the currently approved agents on the market which afford only transient, symptomatic benefits to the patient rather than halting, slowing, or reversing the disease, there is a significant unmet need in the treatment of Alzheimer's disease.

Alzheimer's disease is characterized by the generation, aggregation, and deposition of Abeta in the brain. Complete or partial inhibition of beta-secretase (beta-site amyloid precursor protein-cleaving enzyme; BACE) has been shown to have a significant effect on plaque-related and plaque-dependent pathologies in mouse models suggesting that even small reductions in Abeta peptide levels might result in a long-term significant reduction in plaque burden and synaptic deficits, thus providing significant therapeutic benefits, particularly in the treatment of Alzheimer's disease.

U.S. Pat. No. 8,198,269 discloses certain fused aminodihydrothiazine derivatives which have amyloid-beta protein production inhibitory effect or a BACE1 inhibitory effect and are effective for treating neurodegenerative disease caused by Abeta protein, in particular Alzheimer-type dementia, Down's syndrome or the like. In addition, U.S. Pat. No. 8,822,456 discloses certain hexahydropyrano[3,4-D][1,3]thiazin-2-amine that are inhibitors of BACE1.

The present invention provides certain novel compounds that are inhibitors of BACE1. In addition, the present invention provides certain novel compounds which penetrate the CNS.

Accordingly, the present invention provides a compound of Formula I:

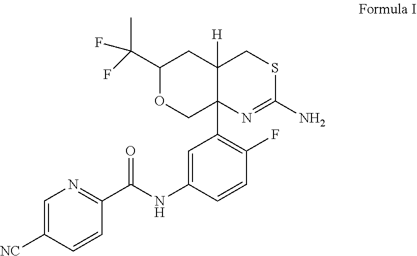 [SMILES: CC(F)(F)C1CC2CSC(N)=NC2(c2cc(NC(=O)c3ccc(C#N)cn3)ccc2F)CO1]

or a pharmaceutically acceptable salt thereof.

In addition, the present invention provides a compound of Formula Ia:

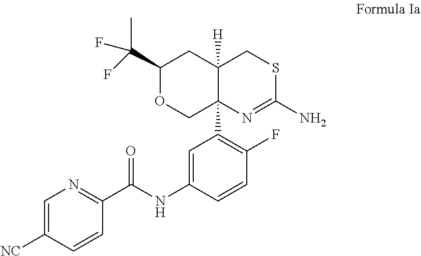 [SMILES: CC(F)(F)[C@H]1C[C@H]2CSC(N)=N[C@@]2(c2cc(NC(=O)c3ccc(C#N)cn3)ccc2F)CO1]

or a pharmaceutically acceptable salt thereof.

The present invention also provides a method of treating Alzheimer's disease in a patient, comprising administering to patient in need of such treatment an effective amount of a compound of Formulas I or Ia, or a pharmaceutically acceptable salt thereof.

The present invention further provides a method of treating the progression of mild cognitive impairment to Alzheimer's disease in a patient, comprising administering to a patient in need of such treatment an effective amount of a compound of Formulas I or Ia, or a pharmaceutically acceptable salt thereof. The present invention also provides a method of inhibiting BACE in a patient, comprising administering to a patient in need of such treatment an effective amount of a compound of Formulas I or Ia, or a pharmaceutically acceptable salt thereof. The present invention also provides a method for inhibiting BACE-mediated cleavage of amyloid precursor protein, comprising administering to a patient in need of such treatment an effective amount of a compound of Formulas I or Ia, or a pharmaceutically acceptable salt thereof. The invention further provides a method for inhibiting production of Abeta peptide, comprising administering to a patient in need of such treatment an effective amount of a compound of Formulas I or Ia, or a pharmaceutically acceptable salt thereof.

Furthermore, this invention provides a compound of Formulas I or Ia, or a pharmaceutically acceptable salt thereof for use in therapy, in particular for use in the treatment of Alzheimer's disease or for use in preventing the progression of mild cognitive impairment to Alzheimer's disease. Even furthermore, this invention provides the use of a compound of Formulas I or Ia, or a pharmaceutically acceptable salt thereof, for the manufacture of a medicament for the treatment of Alzheimer's disease.

The invention further provides a pharmaceutical composition, comprising a compound of Formulas I or Ia, or a pharmaceutically acceptable salt thereof, with one or more pharmaceutically acceptable carriers, diluents, or excipients. The invention further provides a process for preparing a pharmaceutical composition, comprising admixing a compound of Formulas I or Ia, or a pharmaceutically acceptable salt thereof, with one or more pharmaceutically acceptable carriers, diluents, or excipients. This invention also encompasses novel intermediates and processes for the synthesis of the compounds of Formulas I and Ia.

Mild cognitive impairment has been defined as a potential prodromal phase of dementia associated with Alzheimer's disease based on clinical presentation and on progression of patients exhibiting mild cognitive impairment to Alzheimer's dementia over time. (Morris, et al., Arch. Neurol., 58, 397-405 (2001); Petersen, et al., Arch. Neurol., 56, 303-308 (1999)). The term “preventing the progression of mild cognitive impairment to Alzheimer's disease” includes restraining, slowing, stopping, or reversing the progression of mild cognitive impairment to Alzheimer's disease in a patient.

As used herein, the terms “treating” or “to treat” includes restraining, slowing, stopping, or reversing the progression or severity of an existing symptom or disorder.

As used herein, the term “patient” refers to a human.

The term “inhibition of production of Abeta peptide” is taken to mean decreasing in vivo levels of Abeta peptide in a patient.

As used herein, the term “effective amount” refers to the amount or dose of compound of the invention, or a pharmaceutically acceptable salt thereof which, upon single or multiple dose administration to the patient, provides the desired effect in the patient under diagnosis or treatment.

An effective amount can be readily determined by the attending diagnostician, as one skilled in the art, by the use of known techniques and by observing results obtained under analogous circumstances. In determining the effective amount for a patient, a number of factors are considered by the attending diagnostician, including, but not limited to: the species of patient; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

The compounds of the present invention are generally effective over a wide dosage range. For example, dosages per day normally fall within the range of about 0.01 to about 20 mg/kg of body weight. In some instances dosage levels below the lower limit of the aforesaid range may be more than adequate, while in other cases still larger doses may be employed with acceptable side effects, and therefore the above dosage range is not intended to limit the scope of the invention in any way.

The compounds of the present invention are preferably formulated as pharmaceutical compositions administered by any route which makes the compound bioavailable, including oral and transdermal routes. Most preferably, such compositions are for oral administration. Such pharmaceutical compositions and processes for preparing same are well known in the art. (See, e.g., Remington: The Science and Practice of Pharmacy, L. V. Allen, Editor, 22^{nd} Edition, Pharmaceutical Press, 2012).

The compounds of Formulas I and Ia, or pharmaceutically acceptable salts thereof are particularly useful in the treatment methods of the invention, but certain groups, substituents, and configurations are preferred. The following paragraphs describe such preferred groups, substituents, and configurations. It will be understood that these preferences are applicable both to the treatment methods and to the new compounds of the invention.

Further compounds of the present invention include:

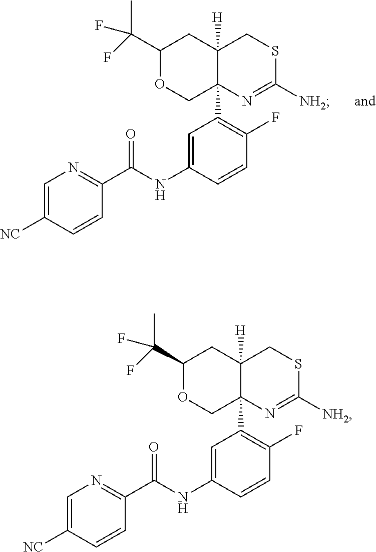 [SMILES: CC(F)(F)C1C[C@H]2CSC(N)=N[C@@]2(c2cc(NC(=O)c3ccc(C#N)cn3)ccc2F)CO1 ; "and" ; CC(F)(F)[C@H]1C[C@H]2CSC(N)=N[C@@]2(c2cc(NC(=O)c3ccc(C#N)cn3)ccc2F)CO1]

and pharmaceutically acceptable salts thereof.

The compound of Formula I wherein the fused bicyclic ring is in the cis configuration, or pharmaceutically acceptable salt thereof, is preferred. For example, one of ordinary skill in the art will appreciate that the hydrogen at position 4a is in the cis configuration relative to the substituted phenyl at position 8a as shown in Scheme A below. In addition, the preferred relative configuration for positions 4a, 6, and 8a are also shown in Scheme A wherein the 1,1-difluoroethyl substituent at position 6 is in the trans configuration relative to the hydrogen at position 4a and the substituted phenyl at position 8a.

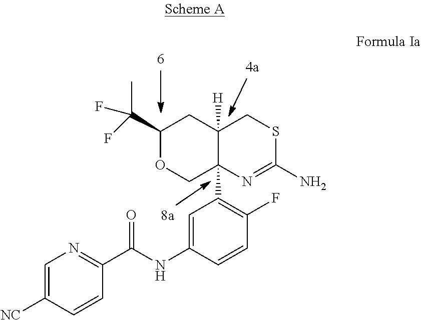 [SMILES: CC(F)(F)[C@H]1C[C@H]2CSC(N)=N[C@@]2(c2cc(NC(=O)c3ccc(C#N)cn3)ccc2F)CO1]

Although the present invention contemplates all individual enantiomers and diasteromers, as well as mixtures of the enantiomers of said compounds, including racemates, the compound with the absolute configuration as set forth below is particularly preferred:

N-[3-[(4aR,6R,8aS)-2-amino-6-(1,1-difluoroethyl)-4a,5,6,8-tetrahydro-4H-pyrano[3,4-d][1,3]thiazin-8a-yl]-4-fluoro-phenyl]-5-cyano-pyridine-2-carboxamide, and pharmaceutically acceptable salts thereof.

One of ordinary skill in the art will appreciate that compounds of the invention can exist in tautomeric forms, as depicted below in Scheme B. When any reference in this application to one of the specific tautomers of the compounds of the invention is given, it is understood to encompass both tautomeric forms and all mixtures thereof.

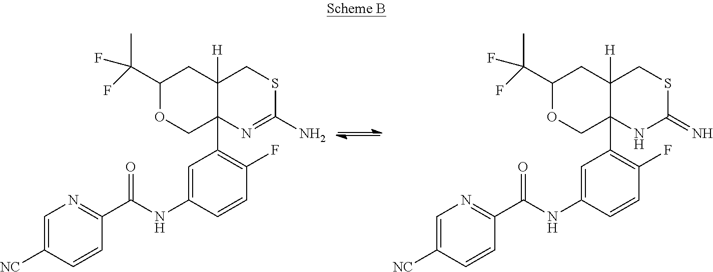 [SMILES: CC(F)(F)C1CC2CSC(N)=NC2(c2cc(NC(=O)c3ccc(C#N)cn3)ccc2F)CO1 ; CC(F)(F)C1CC2CSC(=N)NC2(c2cc(NC(=O)c3ccc(C#N)cn3)ccc2F)CO1]

Additionally, certain intermediates described in the following preparations may contain one or more nitrogen protecting groups. It is understood that protecting groups may be varied as appreciated by one of skill in the art depending on the particular reaction conditions and the particular transformations to be performed. The protection and deprotection conditions are well known to the skilled artisan and are described in the literature (See for example “Greene's Protective Groups in Organic Synthesis”, Fourth Edition, by Peter G. M. Wuts and Theodora W. Greene, John Wiley and Sons, Inc. 2007).

Individual isomers, enantiomers, and diastereomers may be separated or resolved by one of ordinary skill in the art at any convenient point in the synthesis of compounds of the invention, by methods such as selective crystallization techniques or chiral chromatography (See for example, J. Jacques, et al., “Enantiomers. Racemates, and Resolutions”, John Wiley and Sons, Inc., 1981, and E. L. Eliel and S. H. Wilen, “Stereochemistry of Organic Compounds”, Wiley-Interscience, 1994).

A pharmaceutically acceptable salt of the compounds of the invention, such as a hydrochloride salt, can be formed, for example, by reaction of an appropriate free base of a compound of the invention, an appropriate pharmaceutically acceptable acid such as hydrochloric acid in a suitable solvent such as diethyl ether under standard conditions well known in the art. Additionally, the formation of such salts can occur simultaneously upon deprotection of a nitrogen protecting group. The formation of such salts is well known and appreciated in the art. See, for example, Gould, P. L., “Salt selection for basic drugs,” International Journal of Pharmaceutics, 33: 201-217 (1986); Bastin, R. J., et al. “Salt Selection and Optimization Procedures for Pharmaceutical New Chemical Entities,” Organic Process Research and Development, 4: 427-435 (2000); and Berge, S. M., et al., “Pharmaceutical Salts,” Journal of Pharmaceutical Sciences, 66: 1-19, (1977).

Certain abbreviations are defined as follows: “APP” refers to amyloid precursor protein; “ATCC” refers to American Type Culture collection; “BSA” refers to Bovine Serum Albumin; “CDI” refers to 1,1′-carbonyldiimidazole; “cDNA” refers to complementary deoxyribonucleic acid; “DAST” refers to diethylaminosulfur trifluoride; “DCC” refers to 1,3-dicyclohexylcarbodiimide; “Deoxo-Fluor®” refers to bis(2-methoxyethyl)aminosulfur trifluoride; “DIC” refers to 1,3-diisopropylcarbodiimide; “DMAP” refers to 4-dimethylaminopyridine; “DMSO” refers to dimethyl sulfoxide; “EBSS” refers to Earle's Balanced Salt Solution; “EDCI” refers to 1-(3-dimethylaminopropyl)-3-ethylcarbodiimide hydrochloride; “ELISA” refers to enzyme-linked immunosorbent assay; “EtOAc” refers to ethyl acetate; “F12” refers to Ham's F12 medium; “FBS” refers to Fetal Bovine Serum; “Fc” refers to fragment crystallizable; “FLUOLEAD™” refers to 4-tert-butyl-2,6-dimethylphenylsulfur trifluoride; “FRET” refers to fluorescence resonance energy transfer; “HATU” refers to (dimethylamino)-N,N-dimethyl(3H-[1,2,3]triazolo[4,5-b]pyridin-3-yloxy)methaniminium hexafluorophosphate; “HBTU” refers to (1H-benzotriazol-1-yloxy)(dimethylamino)-N,N-dimethylmethaniminium hexafluorophosphate; “HEK” refers to human embryonic kidney; “HF-pyridine” refers to hydrogen fluoride pyridine or Olah's reagent or poly(pyridine fluoride); “HOAt” refers to 1-hydroxy-7-azabenzotriazole; “HOBt” refers to 1-hydroxybenzotriazole hydrate; “hu” refers to human; “IC₅₀” refers to the concentration of an agent that produces 50% of the maximal inhibitory response possible for that agent; “IgG₁” refers to immunoglobulin-like domain Fc-gamma receptor; “MEM” refers to Minimum Essential Medium; “PBS” refers to phosphate buffered saline; “p.o.” refers to orally dosing; “PyBOP” refers to (benzotriazol-1-yl-oxytripyrrolidinophosphonium hexafluorophosphate); “PyBrOP” refers to bromo-tris-pyrrolidino phosphoniumhexafluorophosphate; “RFU” refers to relative fluorescence unit; “RT-PCR” refers to reverse transcription polymerase chain reaction; “SDS-PAGE” refers to sodium dodecyl sulfate polyacrylamide gel electrophoresis; and T3P®” refers to propylphosphonic anhydride; “TEMPO” refers to (2,2,6,6-tetramethyl-piperidin-1-yl)oxyl; “THF” refers to tetrahydrofuran; “Tris” refers to tris(hydroxymethyl)aminomethane; “XtalFluor-E® or DAST difluorosulfinium salt” refers to (diethylamino)difluorosulfonium tetrafluoroborate or N,N-diethyl-S,S-difluorosulfiliminium tetrafluoroborate; and “XtalFluor-M® or morpho-DAST difluorosulfinium salt” refers to difluoro(morpholino)sulfonium tetrafluoroborate or difluoro-4-morpholinylsulfonium tetrafluoroborate.

The compounds of the present invention, or salts thereof, may be prepared by a variety of procedures known to one of ordinary skill in the art, some of which are illustrated in the schemes, preparations, and examples below. One of ordinary skill in the art recognizes that the specific synthetic steps for each of the routes described may be combined in different ways, or in conjunction with steps from different schemes, to prepare compounds of the invention, or salts thereof. The products of each step in the schemes below can be recovered by conventional methods well known in the art, including extraction, evaporation, precipitation, chromatography, filtration, trituration, and crystallization. In the schemes below, all substituents unless otherwise indicated, are as previously defined. The reagents and starting materials are readily available to one of ordinary skill in the art. Without limiting the scope of the invention, the following schemes, preparations, and examples are provided to further illustrate the invention.

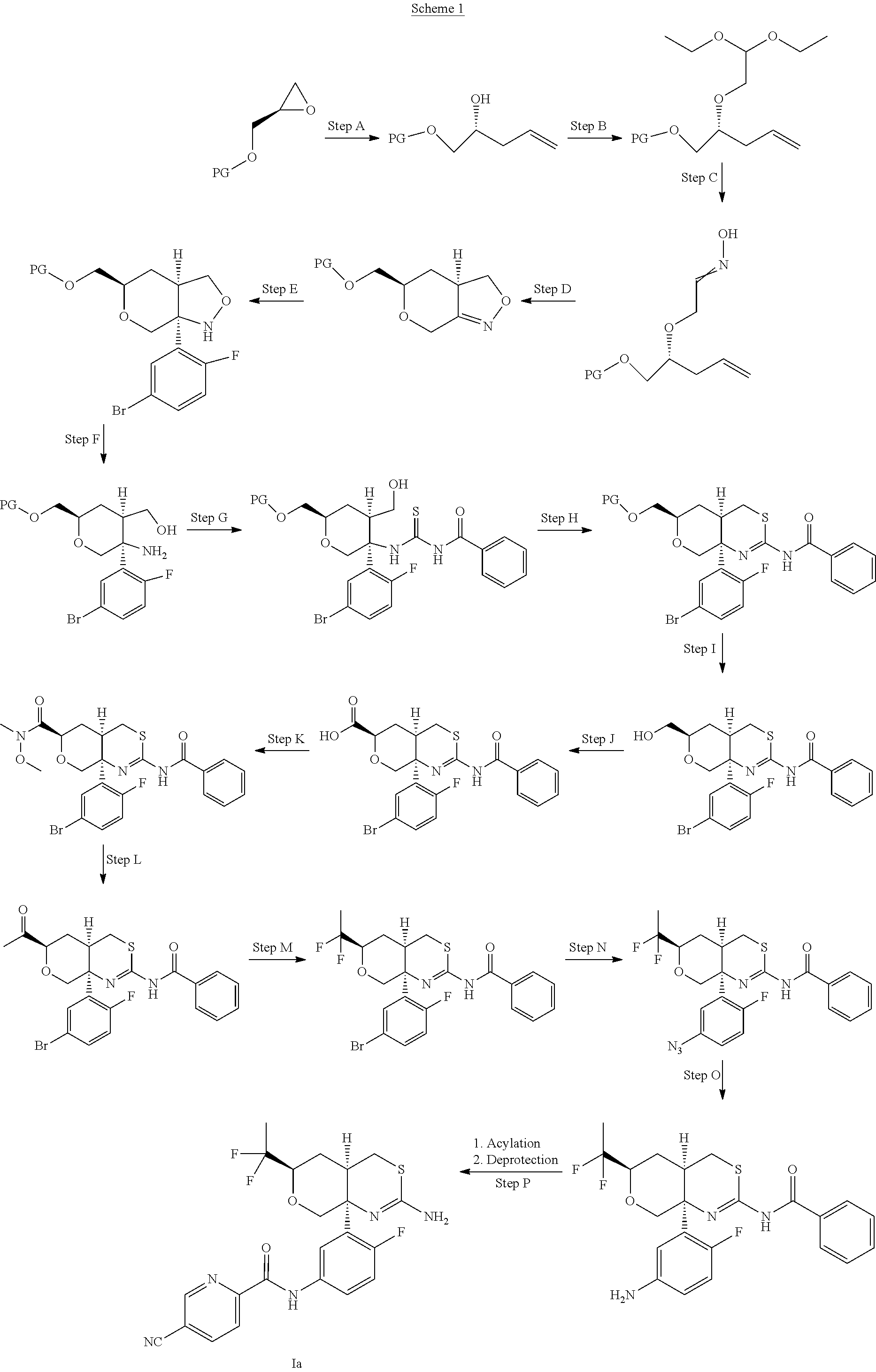

In Scheme 1, Step A, a protected oxymethyl oxirane, protected with a suitable protecting group, such as a benzyl group, is treated with copper iodide in a solvent such as THF and the solution is cooled to about −78° C. The oxirane is alkylated with vinylmagnesium bromide to give the protected product of Scheme 1, Step A. “PG” is a protecting group developed for the amino group or oxygen group such as carbamates, amides, or ethers. Such protecting groups are well known and appreciated in the art. The protected product of Step A is then alkylated at the hydroxy using a strong base such as 60% sodium hydride at about 0° C. in a solvent such as THF or N,N-dimethylformamide.

Adding a halo ether such as bromoacetaldehyde diethylacetal and heating to a temperature of 70-100° C. gives the compound of Scheme 1, Step B. Such alkylations are well known in the art. Alternatively, the protected product of Step A can be reacted with an α-haloester such as tert-butoxy bromoacetate using tetra-N-butylammonium sulfate or other quaternary ammonium salt phase transfer catalysts in a solvent such as toluene and an aqueous inorganic base such as sodium hydroxide at about room temperature to give the protected compound of Step B. The diethoxyethoxy compound of Step B is converted to an oxime over a 2-step procedure. An intermediate aldehyde is formed with the addition of water and formic acid. The reaction is diluted with ethanol and water and treated with sodium acetate followed by hydroxylamine hydrochloride to give the oxime product of Step C. The oxime product of Scheme 1, Step C can be converted to the protected pyrano isoxazole bicyclic product of Step D in a 3+2 cyclization by several methods such as using an aqueous solution of sodium hypochlorite or an alternative oxidant such as N-chlorosuccinimide and in a solvent such as tert-butyl methyl ether, toluene, dichloromethane, or xylene at a temperature of about 10-22° C. or with heating. The 2-fluoro, 5-bromo phenyl group can be added to the pyrano isoxazole by generating the organometallic reagent. The organometallic reagent can be generated from 4-bromo-1-fluoro-2-iodo-benzene using halogen-metal exchange with reagents such as n-butyllithium or isopropylmagnesium chloride lithium chloride complex and dropwise addition at a temperature range of about −78° C. to 15° C. in a solvent such as THF. A Lewis acid such as boron trifluoride diethyl etherate is then added to give the product of Scheme 1, Step E. The resulting bicyclic tetrahydro pyrano isoxazole can be treated with zinc in acetic acid to form the ring opened product of Scheme 1, Step F. An alternate method to open the isoxazole ring uses Raney Nickel in a polar solvent such as ethanol under pressure with hydrogenation conditions. The product of Step F can then be reacted with benzoyl isothiocyanate in a solvent such as dichloromethane or THF at a temperature of about 5° C. to room temperature to give the thiourea compound of Step G. The thiazine ring can be formed using trifluoromethanesulfonic anhydride and an organic base such as pyridine in a solvent such as dichloromethane at a temperature of about −55 to −20° C. to give the product of Step H. The hydroxymethyl protecting group such as a benzyl group can be removed in Scheme 1, Step I using methods well known in the art such as boron trichloride (1 M in dichloromethane) at about 0° C. in a solvent such as dichloromethane to give the compound of Step I. The hydroxy methyl can be oxidized to the carboxylic acid using co-oxidizing agents such as tetrapropylammonium perruthenate and 4-methylmorpholine N-oxide in acetonitrile or alternatively 2-iodoxybenzoic acid (IBX) at temperatures of 0-22° C. in a solvent such as DMSO or addition of (diacetoxyiodo)benzene portionwise or all at once in a solvent such as acetonitrile or acetonitrile and water with stirring at a temperature of about 5-25° C. to give the carboxylic acid of Scheme 1, Step J. TEMPO can also be used as a catalyst in the oxidation. The Weinreb amide is prepared in Scheme 1, Step K from the carboxylic acid of Step J with the addition of N,O-dimethylhydroxylamine hydrochloride, an organic base, such as trimethylamine, and a coupling reagents such as EDCI and HOBt. The mixture is stirred at room temperature to give the product of Step K. Other coupling agents that could be used include CDI, carbodiimides such as DCC, DIC, or other uronium or phosphonium salts of non-nucleophilic anions, such as HBTU, PyBOP, and PyBrOP. The Weinreb amide is then converted to the ketone using an organometallic reagent such as a Grignard reagent or an organolithium reagent in Step L in a solvent such as THF. The appropriate Grignard reagent such as methylmagnesium bromide can be added as a solution in solvents such as ether or 2-methyltetrahydrofuran to the Weinreb amide at a temperature of about −78° C. to 0° C. to give the ketone of Step L. In Scheme 1, Step M, the acetyl group of the compound of Step L can be converted to a difluoro-methyl group using Deoxo-Fluor® in a solvent such as dichloromethane at about −78° C. to room temperature. Another alternative procedure involves pre-mixing the fluorinating reagent such as Deoxo-Fluor® with boron trifluoride-diethyl etherate followed by the addition of the product of Scheme 1, Step L and triethylamine trihydrofluoride to give the product of Scheme 1, Step M. Alternatively, other fluorinating agents that may be used which are well known in the art are, DAST, XtalFluor-E® or XtalFluor-M® with an additive such as triethylamine trihydrofluoride or FLUOLEAD™ using an additive such as HF-pyridine. The 5-bromo of the phenyl is converted to an azide and then to the amine in a step wise procedure (Step N to Step O) using (1R,2R)-N,N′-dimethyl-1,2-cyclohexanediamine or trans-N,N′dimethylcyclohexane-1,2-diamine in a solvent such as ethanol and adding sodium azide followed by sodium L-ascorbate and cupric sulfate. The reaction is heated to about 80-100° C. for several hours or under microwave conditions for a shorter time such as about 90 minutes and then worked up with an extraction using a solvent such as ethyl acetate. The azide product of Step N is then reduced under hydrogenation conditions to the amine using palladium on carbon such as 5-10% palladium in solvents such as methanol or ethanol and THF at a pressure of about 276-345 kPa of hydrogen to give the aniline product of Scheme 1, Step O.

In Scheme 1, Step P, substep 1, the aniline product of Step N can then be acylated under conditions well known in the art with the appropriate carboxylic acid or acid chloride. For example, aniline product of Scheme 1, Step O can be coupled with a heteroaromatic carboxylic acid utilizing coupling conditions well known in the art. One skilled in the art will recognize that there are a number of methods and reagents for amide formation resulting from the reaction of carboxylic acids and amines. For example, the reaction of an appropriate aniline with an appropriate acid in the presence of a coupling reagent and an amine base such as diisopropylethylamine or triethylamine, will give the thiazine protected compound of Scheme 1, Step O, substep 1. Coupling reagents include carbodiimides such as DCC, DIC, EDCI, and aromatic oximes such as HOBt and HOAt. Additionally, uronium or phosphonium salts of non-nucleophilic anions such as HBTU, HATU, PyBOP, and PyBrOP or a cyclic phosphoric anhydride such as T3P® can be used in place of the more traditional coupling reagents. Additives such as DMAP may be used to enhance the reaction. Alternatively, the aniline amine can be acylated using a substituted benzoyl chloride in the presence of a base such as triethylamine or pyridine to give the product of Scheme 1, Step P, substep 1. The thiazine can then be deprotected in Step P, substep 2 under conditions well known in the art using O-methylhydroxylamine hydrochloride in a solvent such as ethanol with an organic base such as pyridine at room temperature or by heating to about 55° C. followed by concentration and purification to give the compound of Formula Ia. Alternatively an inorganic base such as lithium hydroxide in methanol may be used to deprotect the thiazine to give the compound of Formula Ia.

The following Preparations and Examples further illustrate the invention.

### Preparation 1

### (2R)-1-Benzyloxypent-4-en-2-ol

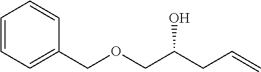

Scheme 1, Step A: Dissolve (R)-benzyloxymethyl-oxirane (ArkPharm, 20 g, 115.7 mmol) in THF (400 mL). Add CuI (1.32 g, 6.94 mmol) and cool to −78° C. then slowly add vinylmagnesium bromide (1 M in THF, 140 mL, 140 mmol) via an addition funnel over about 30 minutes. Stir for 5 hours while allowing the bath to slowly warm to approximately 0° C. then remove the cold bath completely and stir the reaction at room temperature for an additional 30 minutes. Pour the reaction into aq NH₄Cl (˜200 mL) and extract with EtOAc (3×150 mL). Combine the organic extracts, wash with brine, dry over MgSO₄, filter, and concentrate to give the crude product. Purify the material via silica gel chromatography eluting with a 0-25% EtOAc/hexane gradient to give the title compound (21.69 g, 97%). ES/MS m/z 210 [M+H₂O]⁺. See also US2014/0163015.

### Preparation 2

### [(2R)-2-(2,2-Diethoxyethoxy)pent-4-enoxy]methylbenzene

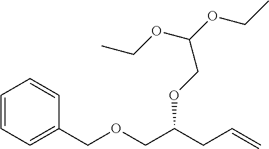 [SMILES: C=CC[C@H](COCc1ccccc1)OCC(OCC)OCC]

Scheme 1, Step B: Dissolve (2R)-1-benzyloxypent-4-en-2-ol (28.9 g, 150 mmol) in THF (500 mL). Cool to 0° C. then carefully add NaH (60% in oil, 9.0 g, 225.6 mmol). Allow to warm to room temperature and stir for 45 minutes then add bromoacetaldehyde diethylacetal (58.3 mL, 376 mmol). Heat to 70° C. for 24 hours. Cool to room temperature. Dilute with EtOAc (150 mL) then pour into 1 N HCl (aq, 100 mL). Separate the layers and extract the aq layer with ethyl acetate (2×150 mL). Combine the organic extracts, wash with brine, dry over MgSO₄, filter, and concentrate to give the crude product. Purify via silica gel chromatography using a 0-25% THF/hexanes gradient to give the title compound (36.08 g, 78%). ES/MS m/z 326 [M+H₂O]⁺. See also US2014/0163015.

### Preparation 3

### 2-[(1R)-1-(Benzyloxymethyl)but-3-enoxy]acetaldehyde oxime

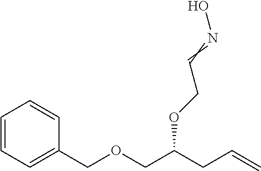 [SMILES: C=CC[C@H](COCc1ccccc1)OCC=NO]

Scheme 1, Step C: Dissolve [(2R)-2-(2,2-diethoxyethoxy)pent-4-enoxy]methylbenzene (6.1 g, 20 mmol) in a mixture of water (8 mL) and formic acid (30 mL). Stir at room temperature for 3 hours to form the intermediate aldehyde. Dilute the reaction solution with ethanol (35 mL) and water (10 mL). Add sodium acetate (4.9 g, 59 mmol) followed by hydroxylamine hydrochloride (4.1 g, 59 mmol). Stir at room temperature for 48 hours. Dilute with EtOAc (50 mL) then pour into saturated aq NaHCO₃ (100 mL) and extract with EtOAc (4×100 mL). Combine the organic extracts, wash with brine, dry over MgSO₄, filter, and concentrate to give the crude product (5.3 g, 110% crude yield) as a mixture of E/Z geometric isomers. The material is used directly without further purification. ES/MS m/z 250 [M+H]. See also US2014/0163015.

### Preparation 4

### (3aR,5R)-5-(benzyloxymethyl)-3a,4,5,7-tetrahydro-3H-pyrano[3,4-c]isoxazole

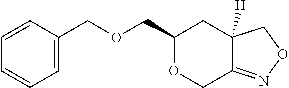 [SMILES: c1ccc(COC[C@H]2C[C@H]3CON=C3CO2)cc1]

Scheme 1, Step D: Dissolve 2-[(1R)-1-(benzyloxymethyl)but-3-enoxy]acetaldehyde oxime (20.8 g, 83.4 mmol) in dichloromethane (300 mL). Add sodium hypochlorite (5% aq, 138 mL, 100 mmol) and stir at room temperature for 24 hours. Pour into water (100 mL) and extract with dichloromethane (2×100 mL). Combine the organic extracts, wash with brine, dry over MgSO₄, filter, and concentrate to give the crude product. Purify the material via silica gel chromatography eluting with gradient of 0-25% THF/hexanes to give the title compound (9.43 g, 46%). ES/MS m/z 248 [M+H]+. See also US2014/0163015.

### Preparation 5

### (3aR,5R,7aS)-5-(Benzyloxymethyl)-7a-(5-bromo-2-fluoro-phenyl)-1,3,3a,4,5,7-hexahydropyrano[3,4-c]isoxazole

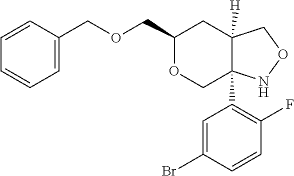 [SMILES: Fc1ccc(Br)cc1[C@]12CO[C@@H](COCc3ccccc3)C[C@H]1CON2]

Scheme 1, Step E: Dissolve 4-bromo-1-fluoro-2-iodobenzene (3.74 mL, 28.4 mmol) in toluene (142 mL). Dilute the solution with THF (14.2 mL) and cool to −78° C. Slowly add n-butyllithium (2.5 M in hexanes, 11 mL, 28.4 mmol). Stir the mixture for 15 minutes and then add borontrifluoride diethyl etherate (3.59 mL, 28.4 mmol). Immediately add a solution of (3aR,5R)-5-(benzyloxymethyl)-3a,4,5,7-tetrahydro-3H-pyrano[3,4-c]isoxazole (3.51 g, 14.2 mmol) in THF (47.3 mL). Stir at −78° C. for 4.5 hours then quench with aq NH₄Cl (50 mL) while still cold. Let the reaction warm to room temperature and stir 30 minutes. Separate the layers and extract the aq layer with EtOAc (2×100 mL). Combine the organic extracts, wash with brine, dry over MgSO₄, filter, and concentrate to give the crude product. Purify the material via silica gel chromatography eluting with a gradient of 0-50% THF/hexanes to give the title compound (3.65 g, 61%). ES/MS m/z (⁷⁹Br/⁸¹Br) 422/424 [M+H]⁺. See also US2014/0163015.

### Preparation 6

### [(2R,4R,5S)-5-Amino-2-(benzyloxymethyl)-5-(5-bromo-2-fluoro-phenyl)tetrahydropyran-4-yl]methanol

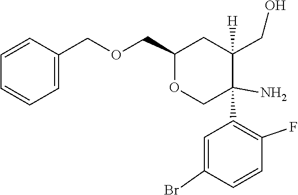 [SMILES: N[C@@]1(c2cc(Br)ccc2F)CO[C@@H](COCc2ccccc2)C[C@H]1CO]

Scheme 1, Step F: Dissolve (3aR,5R,7aS)-5-(benzyloxymethyl)-7a-(5-bromo-2-fluoro-phenyl)-1,3,3a,4,5,7-hexahydropyrano[3,4-c]isoxazole (7.86 g, 18.6 mmol) in acetic acid (250 mL). Add powdered zinc (12.2 g, 186 mmol) and stir at room temperature for 18 hours. Filter the solution through diatomaceous earth, eluting with EtOAc (750 mL). Concentrate the filtrate then dissolve the resulting oil in EtOAc (400 mL). Wash with saturated NaHCO₃ (aq, 2×200 mL) and then with brine. Dry over MgSO₄, filter, and concentrate to give the crude title product (6.43 g, 81%). ES/MS m/z (⁷⁹Br/⁸¹Br) 424/426 [M+H]⁺. See also US2014/0163015.

### Preparation 7

### N-[[(3S,4R,6R)-6-(Benzyloxymethyl)-3-(5-bromo-2-fluoro-phenyl)-4-(hydroxymethyl)tetrahydropyran-3-yl]carbamothioyl]benzamide

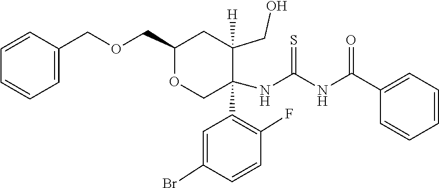 [SMILES: O=C(NC(=S)N[C@@]1(c2cc(Br)ccc2F)CO[C@@H](COCc2ccccc2)C[C@H]1CO)c1ccccc1]

Scheme 1, Step G: Dissolve [(2R,4R,5S)-5-amino-2-(benzyloxymethyl)-5-(5-bromo-2-fluoro-phenyl)tetrahydropyran-4-yl]methanol (12 g, 28.3 mmol) in THF (300 mL). Add benzoyl isothiocyanate (4.58 mL, 33.9 mmol). Stir at room temperature for 18 hours. Pour into saturated NaHCO₃ (aq, 300 mL) and extract with EtOAc (2×300 mL). Concentrate the solution to give the crude product. Purify via silica gel chromatography eluting with a 0-25-50% EtOAc/hexanes step gradient to give the title compound (10.86 g, 65%). ES/MS m/z (⁷⁹Br/⁸¹Br) 587/589 [M+H]⁺. See US2014/0163015.

### Preparation 8

### N-[(4aR,6R,8aS)-6-(Benzyloxymethyl)-8a-(5-bromo-2-fluoro-phenyl)-4a,5,6,8-tetrahydro-4H-pyrano[3,4-d][1,3]thiazin-2-yl]benzamide

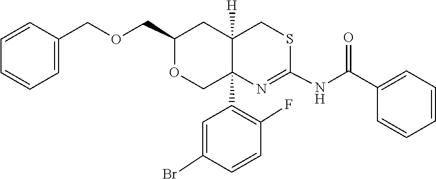 [SMILES: O=C(NC1=N[C@@]2(c3cc(Br)ccc3F)CO[C@@H](COCc3ccccc3)C[C@H]2CS1)c1ccccc1]

Scheme 1, Step H: Dissolve N-[[(3S,4R,6R)-6-(benzyloxymethyl)-3-(5-bromo-2-fluoro-phenyl)-4-(hydroxymethyl)tetrahydropyran-3-yl]carbamothioyl]benzamide (10.86 g, 18.5 mmol) in dichloromethane (125 mL). Cool to −55° C. Add pyridine (5.68 mL, 70.2 mmol) followed by slow addition of trifluoromethanesulfonic anhydride (6.23 mL, 37.0 mmol). Stir the solution for 3 hours while allowing the cold bath to slowly warm then remove the cold bath completely and let the reaction warm to room temperature. Pour into saturated NaHCO₃ (aq, 300 mL) and extract with dichloromethane (2×300 mL). Combine the organic extracts, wash with brine, dry over MgSO₄, filter, and concentrate to give the crude product. Purify via silica gel chromatography eluting with a 0-50% THF/hexanes gradient to give the title compound (8.9 g, 85%). ES/MS m/z (⁷⁹Br/⁸¹Br) 569/571 [M+H]⁺. See US2014/0163015.

### Preparation 9

### N-[(4aR,6R,8aS)-8a-(5-Bromo-2-fluoro-phenyl)-6-(hydroxymethyl)-4a,5,6,8-tetrahydro-4H-pyrano[3,4-d][1,3]thiazin-2-yl]benzamide

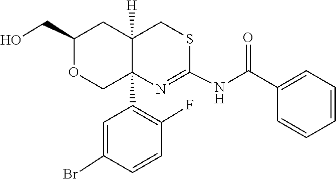 [SMILES: O=C(NC1=N[C@@]2(c3cc(Br)ccc3F)CO[C@@H](CO)C[C@H]2CS1)c1ccccc1]

See US2014/0163015. Scheme 1, Step I: Dissolve N-[(4aR,6R,8aS)-6-(benzyloxymethyl)-8a-(5-bromo-2-fluoro-phenyl)-4a,5,6,8-tetrahydro-4H-pyrano[3,4-d][1,3]thiazin-2-yl]benzamide (8.9 g, 15.7 mmol) in dichloromethane (100 mL). Cool to 0° C. then add boron trichloride (1 M in dichloromethane, 31.4 mL, 31.4 mmol). Stir for 15 minutes then allow to warm to room temperature and stir for 3 hours. Quench the reaction by adding methanol (50 mL) while the reaction is kept under a nitrogen stream. Remove the nitrogen stream and warm to 50° C. with stirring for 30 minutes then return to room temperature and let stand overnight. Concentrate the solution to give the crude product. Purify the material via silica gel chromatography eluting with a 0-100% EtOAc/hexanes gradient to give the title compound (6.9 g, 92%). ES/MS m/z (⁷⁹Br/⁸¹Br) 479/481 [M+H]⁺. See US2014/0163015.

### Preparation 10

### (4aR,6R,8aS)-2-Benzamido-8a-(5-bromo-2-fluoro-phenyl)-4a,5,6,8-tetrahydro-4H-pyrano[3,4-d][1,3]thiazine-6-carboxylic acid

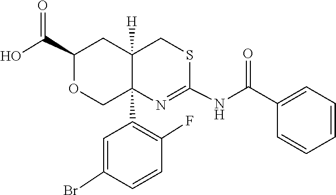 [SMILES: O=C(NC1=N[C@@]2(c3cc(Br)ccc3F)CO[C@@H](C(=O)O)C[C@H]2CS1)c1ccccc1]

See US2014/0163015. Scheme 1, Step J: Dissolve N-[(4aR,6R,8aS)-8a-(5-bromo-2-fluoro-phenyl)-6-(hydroxymethyl)-4a,5,6,8-tetrahydro-4H-pyrano[3,4-d][1,3]thiazin-2-yl]benzamide (6.9 g, 14.4 mmol) in acetonitrile (100 mL). Add 4-methylmorpholine N-oxide (10.4 g, 86.5 mmol) followed by tetrapropylammonium perruthenate (0.52 g, 1.44 mmol). Stir at room temperature for 5 hours then let stand overnight. Add isopropanol (30 mL) and stir at room temperature for 30 minutes. Concentrate the solution to give the crude title product (5.46 g, 77%). ES/MS m/z (⁷⁹Br/⁸¹Br) 493/495 [M+H]⁺. See US2014/0163015.

### Preparation 11

### (4aR,6R,8aS)-2-Benzamido-8a-(5-bromo-2-fluoro-phenyl)-N-methoxy-N-methyl-4a,5,6,8-tetrahydro-4H-pyrano[3,4-d][1,3]thiazine-6-carboxamide

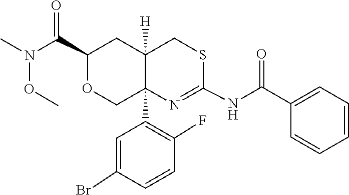 [SMILES: CON(C)C(=O)[C@H]1C[C@H]2CSC(NC(=O)c3ccccc3)=N[C@@]2(c2cc(Br)ccc2F)CO1]

Scheme 1, Step K: Add together (4aR,6R,8aS)-2-benzamido-8a-(5-bromo-2-fluoro-phenyl)-4a,5,6,8-tetrahydro-4H-pyrano[3,4-d][1,3]thiazine-6-carboxylic acid (5.46 g, 11.1 mmol) and dichloromethane (100 mL). Add the following to the reaction: N,O-dimethylhydroxylamine hydrochloride (1.65 g, 16.6 mmol), HOBt (2.59 g, 18.8 mmol), EDCI (3.18 g, 16.6 mmol) and trimethylamine (4.63 mL, 33.2 mmol). Stir at room temperature for 18 hours. If by LC/MS starting material remains, repeat addition of the reagents in the same amounts and stir another 24 hours. Once the starting material is consumed as indicated by LC/MS, pour the reaction into saturated NaHCO₃ (aq, 300 mL) and extract the solution with dichloromethane (2×300 mL). Combine the organic extracts, wash with brine, dry over MgSO₄, filter, and concentrate the solution to give the crude product. Purify the material via silica gel chromatography eluting with a 0-100% EtOAc/hexanes gradient to give the title compound (4 g, 67%). ES/MS m/z (⁷⁹Br/⁸¹Br) 536/538 [M+H]⁺.

### Preparation 12

### N-[(4aR,6R,8aS)-6-Acetyl-8a-(5-bromo-2-fluoro-phenyl)-4a,5,6,8-tetrahydro-4H-pyrano[3,4-d][1,3]thiazin-2-yl]benzamide

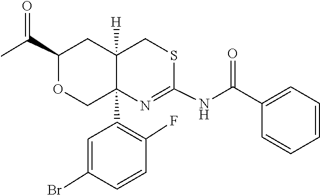 [SMILES: CC(=O)[C@H]1C[C@H]2CSC(NC(=O)c3ccccc3)=N[C@@]2(c2cc(Br)ccc2F)CO1]

Scheme 1, Step L: Dissolve (4aR,6R,8aS)-2-benzamido-8a-(5-bromo-2-fluoro-phenyl)-N-methoxy-N-methyl-4a,5,6,8-tetrahydro-4H-pyrano[3,4-d][1,3]thiazine-6-carboxamide (4 g, 7.5 mmol) in THF (100 mL) and cool the mixture to 0° C. Add methylmagnesium bromide (3 M in diethyl ether, 7.5 mL, 22.4 mmol) and stir for 5 hours while allowing the reaction and ice bath to slowly warm to room temperature. Pour the reaction into saturated, aq NH₄Cl (300 mL) and extract with EtOAc (2×200 mL). Combine the organic extracts, wash with brine, dry over MgSO₄, filter, and concentrate the solution to give the crude product. Purify the material via silica gel chromatography eluting with a 0-50% EtOAc/hexanes gradient to give the title compound (2.77 g, 76%). ES/MS m/z (⁷⁹Br/⁸¹Br) 491/493 [M+H]⁺.

### Preparation 13

### N-[(4aR,6R,8aS)-8a-(5-Bromo-2-fluoro-phenyl)-6-(1,1-difluoroethyl)-4a,5,6,8-tetrahydro-4H-pyrano[3,4-d][1,3]thiazin-2-yl]benzamide

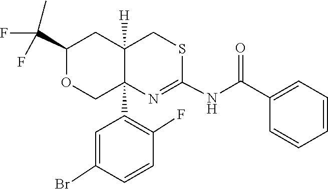 [SMILES: CC(F)(F)[C@H]1C[C@H]2CSC(NC(=O)c3ccccc3)=N[C@@]2(c2cc(Br)ccc2F)CO1]

Scheme 1, Step M: Dissolve N-[(4aR,6R,8aS)-6-acetyl-8a-(5-bromo-2-fluoro-phenyl)-4a,5,6,8-tetrahydro-4H-pyrano[3,4-d][1,3]thiazin-2-yl]benzamide (2.77 g, 5.64 mmol) in dichloromethane (200 mL). Add Deoxo-Fluor® (50% in THF, 9.59 ml, 22.5 mmol). Stir at room temperature for 36 hours. Pour the reaction into saturated, aq NaHCO₃ (300 mL) and extract the solution with dichloromethane (2×200 mL). Combine the organic extracts, wash with brine, dry over MgSO₄, filter, and concentrate to give the crude product. Purify the material via silica gel chromatography eluting with a 0-25-50% EtOAc/hexanes gradient to give the title compound (1.68 g, 58%). ES/MS m/z (⁷⁹Br/⁸¹Br) 513/515 [M+H]⁺.

### Preparation 14

### N-[(4aR,6R,8aS)-8a-(5-Azido-2-fluoro-phenyl)-6-(1,1-difluoroethyl)-4a,5,6,8-tetrahydro-4H-pyrano[3,4-d][1,3]thiazin-2-yl]benzamide

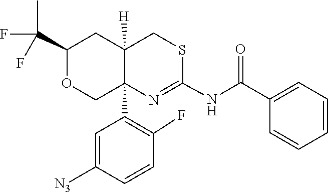 [SMILES: CC(F)(F)[C@H]1C[C@H]2CSC(NC(=O)c3ccccc3)=N[C@@]2(c2cc(N=[N+]=[N-])ccc2F)CO1]

Scheme 1, Step N: Dissolve N-[(4aR,6R,8aS)-8a-(5-bromo-2-fluoro-phenyl)-6-(1,1-difluoroethyl)-4a,5,6,8-tetrahydro-4H-pyrano[3,4-d][1,3]thiazin-2-yl]benzamide (600 mg, 1.17 mmol) in ethanol (12 mL). Add (1R,2R)-N,N′-dimethyl-1,2-cyclohexanediamine (0.06 mL, 0.35 mmol). Degas by bubbling nitrogen through the reaction mixture for 10 minutes and add sodium azide (0.30 g, 4.68 mmol). Add a freshly prepared solution of sodium ascorbate (0.66 M in water, 0.78 mL, 0.51 mmol) followed by a freshly prepared solution of cupric sulfate (0.33 M in water, 1.1 mL, 0.35 mmol). Seal the reaction and heat via microwave irradiation to 80° C. for 90 minutes. Pour the reaction into saturated, aq NaHCO₃ (250 mL). Extract the mixture with EtOAc (2×100 mL). Combine the organic extracts, wash with brine, dry over MgSO₄, filter, and concentrate to give the crude product (700 mg, 130%). ES/MS m/z 476 [M+H]⁺. Carry the crude mixture forward without further purification. Note that the crude product may also contain a significant amount of N-[(4aR,6R,8aS)-8a-(5-amino-2-fluoro-phenyl)-6-(1,1-difluoroethyl)-4a,5,6,8-tetrahydro-4H-pyrano[3,4-d][1,3]thiazin-2-yl]benzamide.

### Preparation 15

### N-[(4aR,6R,8aS)-8a-(5-Amino-2-fluoro-phenyl)-6-(1,1-difluoroethyl)-4a,5,6,8-tetrahydro-4H-pyrano[3,4-d][1,3]thiazin-2-yl]benzamide

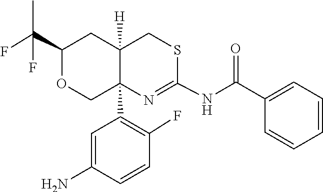 [SMILES: CC(F)(F)[C@H]1C[C@H]2CSC(NC(=O)c3ccccc3)=N[C@@]2(c2cc(N)ccc2F)CO1]

Scheme 1, Step O: Dissolve N-[(4aR,6R,8aS)-8a-(5-azido-2-fluoro-phenyl)-6-(1,1-difluoroethyl)-4a,5,6,8-tetrahydro-4H-pyrano[3,4-d][1,3]thiazin-2-yl]benzamide (0.56 g, 1.17 mmol in methanol (30 mL). Add 10% Pd on carbon (100 mg). Stir at room temperature under H₂ (101 kPa) applied via a balloon of H₂ gas for 6 hours. Filter the solution through diatomaceous earth, eluting with methanol. Concentrate the solution and purify the residue via silica gel chromatography eluting with a 0-50% EtOAc/hexanes gradient to give the title compound (0.48 g, 91%). ES/MS m/z 450 [M+H]⁺.

### Preparation 16

### N-[3-[(4aR,6R,8aS)-2-Benzamido-6-(1,1-difluoroethyl)-4a,5,6,8-tetrahydro-4H-pyrano[3,4-d][1,3]thiazin-8a-yl]-4-fluoro-phenyl]-5-cyano-pyridine-2-carboxamide

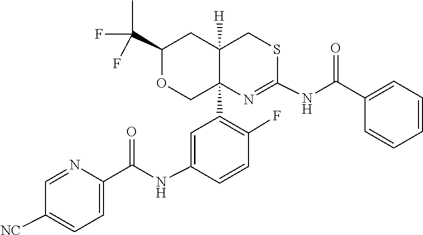 [SMILES: CC(F)(F)[C@H]1C[C@H]2CSC(NC(=O)c3ccccc3)=N[C@@]2(c2cc(NC(=O)c3ccc(C#N)cn3)ccc2F)CO1]

Scheme 1, Step P, substep 1: Dissolve N-[(4aR,6R,8aS)-8a-(5-amino-2-fluoro-phenyl)-6-(1,1-difluoroethyl)-4a,5,6,8-tetrahydro-4H-pyrano[3,4-d][1,3]thiazin-2-yl]benzamide (0.50 g, 1.11 mmol) in dichloromethane (15 ML). Add 5-cyanopyridine-2-carboxylic acid (0.26 g, 1.67 mmol) followed by triethylamine (0.47 mL, 3.34 mmol), HOBt (0.23 g, 1.67 mmol), and EDCI (0.32 g, 1.67 mmol). Stir at room temperature for 24 hours. Pour the reaction mixture into saturated, aq NaHCO₃(150 mL). Extract the solution with EtOAc (2×100 mL). Combine the organic extracts, wash with brine, dry over MgSO₄, filter, and concentrate to give the crude product. Purify the residue via silica gel chromatography eluting with a 0-50% EtOAc/hexanes gradient to give the title compound (0.46 g, 70%). ES/MS m/z 580 [M+H]⁺.

### Example 1

### N-[3-[(4aR,6R,8aS)-2-Amino-6-(1,1-difluoroethyl)-4a,5,6,8-tetrahydro-4H-pyrano[3,4-d][1,3]thiazin-8a-yl]-4-fluoro-phenyl]-5-cyano-pyridine-2-carboxamide

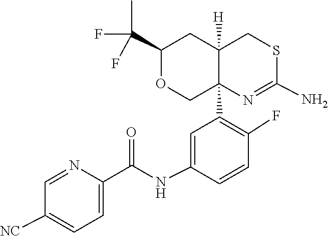 [SMILES: CC(F)(F)[C@H]1C[C@H]2CSC(N)=N[C@@]2(c2cc(NC(=O)c3ccc(C#N)cn3)ccc2F)CO1]

Scheme 1, Step P, substep 2: Dissolve N-[3-[(4aR,6R,8aS)-2-benzamido-6-(1,1-difluoroethyl)-4a,5,6,8-tetrahydro-4H-pyrano[3,4-d][1,3]thiazin-8a-yl]-4-fluoro-phenyl]-5-cyano-pyridine-2-carboxamide (0.46 g, 0.80 mmol) in ethanol (10 mL). Add O-methylhydroxylamine hydrochloride (0.69 g, 8.0 mmol) followed by pyridine (0.65 mL, 8.0 mmol). Stir at room temperature for 22 hours. Concentrate the reaction and purify the residue via silica gel chromatography eluting with a 0-5% (7 N NH₃ in methanol)/dichloromethane gradient to give the title compound (0.355 g, 75%). ES/MS m/z 476 [M+H]⁺.

### In Vitro Assay Procedures:

To assess selectivity of BACE1 over BACE2, the test compound is evaluated in FRET assays using specific substrates for BACE1 and BACE2 as described below. For in vitro enzymatic and cellular assays, the test compound is prepared in DMSO to make up a 10 mM stock solution. The stock solution is serially diluted in DMSO to obtain a ten-point dilution curve with final compound concentrations ranging from 10 μM to 0.05 nM in a 96-well round-bottom plate before conducting the in vitro enzymatic and whole cell assays.

### In Vitro Protease Inhibition Assays:

### Expression of huBACE1:Fc and huBACE2:Fc

Human BACE1 (accession number: AF190725) and human BACE2 (accession number: AF 204944) are cloned from total brain cDNA by RT-PCR. The nucleotide sequences corresponding to amino acid sequences #1 to 460 are inserted into the cDNA encoding human IgG₁ (Fc) polypeptide (Vassar et al., Science, 286, 735-742 (1999)). This fusion protein of BACE1(1-460) or BACE2(1-460) and human Fc, named huBACE1:Fc and huBACE2:Fc respectively, are constructed in the pJB02 vector. Human BACE1(1-460):Fc (huBACE1:Fc) and human BACE2(1-460):Fc (huBACE2:Fc) are transiently expressed in HEK293 cells. cDNA (250 μg) of each construct are mixed with Fugene 6 and added to 1 liter HEK293 cells. Four days after the transfection, conditioned media are harvested for purification. huBACE1:Fc and huBACE2:Fc are purified by Protein A chromatography as described below. The enzymes are stored at −80° C. in small aliquots. (See Yang, et. al., J. Neurochemistry, 91(6) 1249-59 (2004).

### Purification of huBACE1:Fc and huBACE2:Fc

Conditioned media of HEK293 cells transiently transfected with huBACE1:Fc or huBACE2:Fc cDNA are collected. Cell debris is removed by filtering the conditioned media through 0.22 m sterile filter. Protein A-agarose (5 ml) (bed volume) is added to conditioned media (4 liter). This mixture is gently stirred overnight at 4° C. The Protein A-agarose resin is collected and packed into a low-pressure chromatography column. The column is washed with 20× bed volumes of PBS at a flow rate 20 ml per hour. Bound huBACE1:Fc or huBACE2:Fc protein is eluted with 50 mM acetic acid, pH 3.6, at flow rate 20 ml per hour. Fractions (1 ml) of eluent are neutralized immediately with ammonium acetate (0.5 ml, 200 mM), pH 6.5. The purity of the final product is assessed by electrophoresis in 4-20% Tris-Glycine SDS-PAGE. The enzyme is stored at −80° C. in small aliquots.

### BACE1 FRET Assay

Serial dilutions of the test compound are prepared as described above. The compound is further diluted 20× in KH₂PO₄ buffer. Each dilution (10 μL) is added to each well on row A to H of a corresponding low protein binding black plate containing the reaction mixture (25 μL of 50 mM KH₂PO₄, pH 4.6, 1 mM TRITON® X-100, 1 mg/mL BSA, and 15 μM of FRET substrate based upon the sequence of APP) (See Yang, et. al., J. Neurochemistry, 91(6) 1249-59 (2004)). The content is mixed well on a plate shaker for 10 minutes. Human BACE1(1-460):Fc (15 μL of 200 μM) (See Vasser, et al., Science, 286, 735-741 (1999)) in the KH₂PO₄ buffer is added to the plate containing substrate and the test compound to initiate the reaction. The RFU of the mixture at time 0 is recorded at excitation wavelength 355 nm and emission wavelength 460 nm, after brief mixing on a plate shaker. The reaction plate is covered with aluminum foil and kept in a dark humidified oven at room temperature for 16 to 24 hours. The RFU at the end of incubation is recorded with the same excitation and emission settings used at time 0. The difference of the RFU at time 0 and the end of incubation is representative of the activity of BACE1 under the compound treatment. RFU differences are plotted versus inhibitor concentration and a curve is fitted with a four-parameter logistic equation to obtain the IC₅₀ value. (May, et al., Journal of Neuroscience, 31, 16507-16516 (2011)).

The compound of Example 1 is tested essentially as described above and exhibits an IC₅₀ for BACE1 of 0.263 nM±0.035, n=5 (Mean±standard deviation of the mean). This data demonstrates that the compound of Example 1 inhibits purified recombinant BACE1 enzyme activity in vitro.

### SH-SY5YAPP695Wt Whole Cell Assay

The routine whole cell assay for the measurement of inhibition of BACE1 activity utilizes the human neuroblastoma cell line SH-SY5Y (ATCC Accession No. CRL2266) stably expressing a human APP695Wt cDNA. Cells are routinely used up to passage number 6 and then discarded.

SH-SY5YAPP695Wt cells are plated in 96 well tissue culture plates at 5.0×10⁴ cells/well in 200 μL culture media (50% MEM/EBSS and Ham's F12, 1× each sodium pyruvate, non-essential amino acids and NaHCO₃ containing 10% FBS). The following day, media is removed from the cells, fresh media added then incubated at 37° C. for 24 hours in the presence/absence of test compound at the desired concentration range.

At the end of the incubation, conditioned media are analyzed for evidence of beta-secretase activity by analysis of Abeta peptides 1-40 and 1-42 by specific sandwich ELISAs. To measure these specific isoforms of Abeta, monoclonal 2G3 is used as a capture antibody for Abeta 1-40 and monoclonal 21F12 as a capture antibody for Abeta 1-42. Both Abeta 1-40 and Abeta 1-42 ELISAs use biotinylated 3D6 as the reporting antibody (for description of antibodies, see Johnson-Wood, et al., Proc. Natd. Acad. Sci. USA 94, 1550-1555 (1997)). The concentration of Abeta released in the conditioned media following the compound treatment corresponds to the activity of BACE1 under such conditions. The 10-point inhibition curve is plotted and fitted with the four-parameter logistic equation to obtain the IC₅₀ values for the Abeta-lowering effect.

The compound of Example 1 is tested essentially as described above and exhibits an IC₅₀ of 0.0260 nM±0.0104, n=6 for SH-SY5YAPP695Wt A-beta (1-40) ELISA and an IC₅₀ of 0.0313 nM±0.0187, n=6 for SH-SY5YAPP695Wt A-beta (1-42) ELISA (Mean±standard deviation of the mean). The data set forth above demonstrates that the compound of Example 1 inhibits BACE1 in the whole cell assay.

1. A compound of the formula: 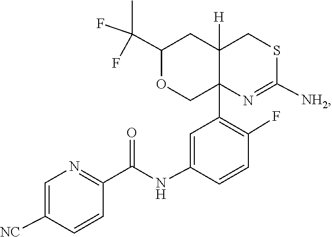 [SMILES: CC(F)(F)C1CC2CSC(N)=NC2(c2cc(NC(=O)c3ccc(C#N)cn3)ccc2F)CO1]
or a pharmaceutically acceptable salt thereof.

2. The compound or salt according to claim 1 wherein the hydrogen at position 4a is in the cis configuration relative to the substituted phenyl at position 8a:

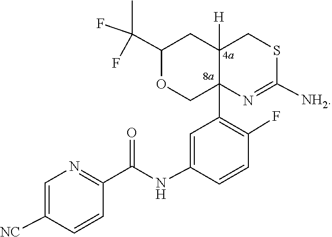 [SMILES: CC(F)(F)C1CC2CSC(N)=NC2(c2cc(NC(=O)c3ccc(C#N)cn3)ccc2F)CO1]

3. The compound or salt according to claim 2 wherein the 1,1-difluoroethyl at position 6 is in the trans configuration relative to the hydrogen at position 4a and the substituted phenyl at position 8a:

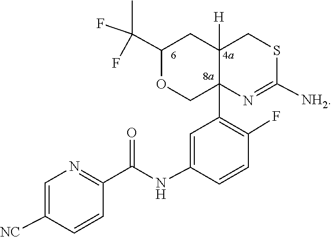 [SMILES: CC(F)(F)C1CC2CSC(N)=NC2(c2cc(NC(=O)c3ccc(C#N)cn3)ccc2F)CO1]

4. A compound which is N-[3-[(4aR,6R,8aS)-2-amino-6-(1,1-difluoroethyl)-4a,5,6,8-tetrahydro-4H-pyrano[3,4-d][1,3]thiazin-8a-yl]-4-fluoro-phenyl]-5-cyano-pyridine-2-carboxamide, or a pharmaceutically acceptable salt thereof.

5. A method of treating Alzheimer's disease in a patient, comprising administering to a patient in need of such treatment an effective amount of a compound according to claim 1, or a pharmaceutically acceptable salt thereof.

6. A method of preventing the progression of mild cognitive impairment to Alzheimer's disease in a patient, comprising administering to a patient in need of such treatment an effective amount of a compound according to claim 1, or a pharmaceutically acceptable salt thereof.

7. (canceled)

8. (canceled)

9. (canceled)

10. A pharmaceutical composition, comprising a compound or a pharmaceutically acceptable salt thereof according to claim 1 with one or more pharmaceutically acceptable carriers, diluents, or excipients.

11. A process for preparing a pharmaceutical composition, comprising admixing a compound or a pharmaceutically acceptable salt thereof according to claim 1 with one or more pharmaceutically acceptable carriers, diluents, or excipients.

12. A method of treating Alzheimer's disease in a patient, comprising administering to a patient in need of such treatment an effective amount of a compound according to claim 4, or a pharmaceutically acceptable salt thereof.

13. A method of preventing the progression of mild cognitive impairment to Alzheimer's disease in a patient, comprising administering to a patient in need of such treatment an effective amount of a compound according to claim 4, or a pharmaceutically acceptable salt thereof.

14. A pharmaceutical composition, comprising a compound or a pharmaceutically acceptable salt thereof according to claim 4 with one or more pharmaceutically acceptable carriers, diluents, or excipients.
